(19) United States
(12) Patent Application Publication
(10) Pub. No.: US 2024/0209410 A1
(43) Pub. Date: Jun. 27, 2024

(54) METHOD FOR THE HYDROXYLATION OF STEROIDS

(71) Applicants: ANNIKKI GMBH, Raaba-Grambach (AT); PHARMAZELL GMBH, Raubling (DE)

(72) Inventors: Nicole Staunig, Vasoldsberg (AT); Kai Oliver Donsbach, Traunstein (DE)

(21) Appl. No.: 17/909,727

(22) PCT Filed: Mar. 5, 2021
(86) PCT No.: PCT/EP2021/055615

§ 371 Date: Sep. 6, 2022

(30) Foreign Application Priority Data
Mar. 6, 2020 (EP) 20161537.4

(51) Int. Cl. C12P 33/06; C12N 9/02; C12N 9/04

(52) U.S. Cl. CPC C12P 33/06; C12N 9/0006; C12N 9/0071; C12Y 101/01; C12Y 114/14

(57) ABSTRACT

The present invention relates to a method of preparing a steroid comprising the step of converting a 7-deoxysteroid with a cytochrome P450 enzyme or a functional variant thereof in the presence of at least one redox partner system and a system for regenerating the redox partner system.

### TECHNICAL FIELD

The present invention relates to means and methods for the hydroxylation of steroids.

### BACKGROUND OF THE INVENTION

3,7,12-Trihydroxylated bile acids, such as, e.g., cholic acid (3α,7α,12α-trihydroxy-50-cholanic acid) or ursocholic acid (3α,7β,12α-trihydroxy-5β-cholanic acid), are industrially important chemicals, among other things as starting materials for the manufacture of ursodeoxycholic acid (UDCA). Ursodeoxycholic acid is used, among other things, as a medicament for dissolving minor X-ray negative gallstones as well as for treating the liver diseases primary ciliary cirrhosis and primary sclerosing cholangitis.

The industrially most important source of 3,7,12-trihydroxylated bile acids is biliary fluid from gallbladders, which accumulate as slaughterhouse waste in meat production. Besides other animal species, the bile of cattle is often used. There is no industrially relevant total synthesis for 3,7,12-trihydroxylated bile acids. Since the production of bile acids is linked to another product (meat), the response to increased demands can only be very limited. For this reason, it is of great interest to use the raw material bile as efficiently as possible.

Since bile is an aqueous mixture of bile acids, lipids, cholesterol and other substances, the separation of the components during the extraction of bile acids is of particular importance. The bile acids, in turn, also constitute a mixture the components of which differ in the number and position of the hydroxyl groups. In addition to cholic acid, bovine bile also contains a significant proportion of deoxycholic acid, which differs from cholic acid in that the OH group is missing at position 7 (3α,12α-dihydroxy-5ß-cholanic acid). Deoxycholic acid has a much lower commercial value than 3,7,12-trihydroxylated bile acids. Therefore, there is an industrial interest in converting deoxycholic acid into a 3,7,12-trihydroxylated bile acid by selectively introducing a hydroxyl group at position 7.

During hydroxylations, an oxygen atom is formally introduced into a (non-activated) C—H bond in an oxidation reaction. In organic chemistry, these are reactions that are very difficult to perform. OH groups are frequently introduced through detours, e.g., by adding water at a C═C double bond. The selective hydroxylation at a specific position of a complex molecule (such as, e.g., a bile acid) is problematic, since several chemically (almost) equivalent C—H bonds are present.

A method for the enzymatic conversion of lithocholic acid to ursodeoxycholic acid using a 70-hydroxylase is disclosed in WO 2018/227940 A1.

Schmitz et al. (Microbial Cell Factories 13 (1): 1-13 (2014)) describe the production of 7-hydroxyl derivatives of dehydroepiandrosterone (DHEA) or pregnenolone (PREG) by conversion with a cytochrome P450 monooxygenase.

CN 102 002 518 B discloses the conversion of 3-β-cholesterol acetate to 7-β-hydroxyl-3-β-cholesterol acetate by means of a hydroxylase.

In WO 2020/109776 A2, a method for the hydroxylation or dealkylation of various organic compounds, among other things from the steroid pregnenolone, with a cytochrome P450 is disclosed.

It is an object of the present invention to provide means and methods of hydroxylating steroids, such as bile acids and derivatives thereof, which have a hydrogen and no hydroxyl group at position 7, specifically at this point.

### SUMMARY OF THE INVENTION

The object according to the invention is achieved by a method of preparing a steroid having the general formula (I)

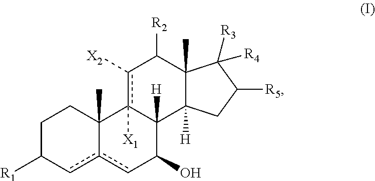

wherein
X₁ and X₂ are independently H, Cl, F, Br, I, CF₃, a C₁ to C₆ alkyl radical, OH, a C₁ to C₆ alkoxy radical, CN, NO₂, N(R₆)₂, an epoxy group, CHO or a CO₂R₆ radical, wherein
   R₆ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH₂(CH₃)₂, —C(O)C(CH₃)₃, C(O)Ph, —C(O)CH₂Ph,
R₁ and R₂ are independently H, OH, OR₇ or O, wherein
   R₇ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH₂(CH₃)₂, —C(O)C(CH₃)₃, —C(O)Ph, —C(O)CH₂Ph,
R₃ is H, OH, OR₈, a C₁ to C₁₀ alkyl radical, a C₁ to C₁₀ alkenyl radical, —CHO, —C(O)(CH₃), —C(O)(CH₂OH), —CH(CH₃)C(O)CH₃, —CH(CH₃)((CH₂)₂CO₂R₉) or —CH(CH₃)((CH₂)₂CONHR₉), wherein
   R₈ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH₂(CH₃)₂, —C(O)C(CH₃)₃, —C(O)Ph or —C(O)CH₂Ph, and
   R₉ is —CH₃, —CH₂COOH, —CH₂CH₃, —CH(CH₃)₂, —(CH₂)₂CH₃, —(CH₂)₂SO₃H, C(CH₃)₃, —(CH₂)₃CH₃, —CH(CH₃)CH₂CH₃, —CH₂CH₂(CH₃)₂, an aryl group or an alkylaryl group,
R₄ is H, OH, or —OR₁₀, wherein
   R₁₀ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂C₁₃, —C(O)CH₂CH₂(CH₃)₂, —C(O)C(CH₃)₃, —C(O)Ph or —C(O)CH₂Ph, and
R₅ is H, CF₃, a C₁ to C₆ alkyl radical, a C₁ to C₆ alkenyl radical, OH, O, or a C₁ to C₆ alkoxy radical, wherein the dashed line denotes an optional double bond, with the proviso that the B ring has no double bond if the A ring has a C4-C5 double bond, and the C ring has no double bond if X₁ and X₂ form an epoxy group, comprising the step of converting a 7-deoxysteroid having the general formula (II)

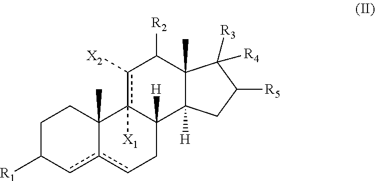

with a cytochrome P450 hydroxylase or a functional variant thereof in the presence of at least one redox partner system and a system for regenerating the redox partner system, the cytochrome P450 enzyme comprising an amino acid sequence which is at least 90%, in particular 100%, identical to the amino acid sequence SEQ ID No. 1 or 2.

Surprisingly, it has been shown that cytochrome P450 and functional fragments thereof are capable of hydroxylating steroids such as cholic acid and, respectively, derivatives thereof having the formula (I) at position 7. By coupling this reaction with at least one redox partner system and a system for regenerating the redox partner system, the equilibrium of the reaction can be shifted towards the end product and the yield thereof can thus be increased significantly. In this case, the regeneration of the redox partner system can take place in the presence of a second system (a regeneration system) which preferably comprises at least one oxidoreductase and at least one substrate of the at least one oxidoreductase.

### DESCRIPTION OF THE EMBODIMENTS

Cytochrome P450 and functional variants thereof, which require the oxidation of reducing equivalents NAD(P)H, are surprisingly capable of selectively hydroxylating 7-deoxysteroids, such as, e.g., 7-deoxycholic acid, and derivatives thereof at position 7.

According to the present invention, the cytochrome P450 enzyme comprises an amino acid sequence which is at least 90%, in particular 100%, identical to the amino acid sequence SEQ ID No. 1 or 2.

Cytochromes P450 catalyze monooxygenase reactions of a large number of endogenous as well as exogenous substrates. They are involved, among other things, in the metabolism of steroids, eicosanoids, fatty acids and bile acids as well as of exogenous substrates such as drugs, insecticides and chemical carcinogens.

Cytochromes P450 according to the present invention can be used, for example, from bacteria such as actinobacteria, in particular, for example, from the genus Streptomyces. In this case, the sequences can be isolated, for example, from genomic DNA or a cDNA library using known techniques.

The cytochromes P450 according to the present invention and, respectively, their functional variants can optionally be present in their original organism or can be isolated therefrom, or they are expressed recombinantly or produced synthetically. Recombinantly expressed polypeptides are preferably used according to the invention.

Various established microorganisms can be used for the recombinant expression of enzymes according to the present invention, such as, e.g., Escherichia coli (E. coli), Bacillus subtilis, Saccharomyces cerevisiae or Pichia pastoris. Appropriate protocols in this regard are described in detail in the relevant specialist literature or are known to a person skilled in the art.

According to the present invention, enzymes/polypeptides are preferably used as proteins recombinantly overexpressed in E. coli, with the corresponding cell lysates preferably being used either without further processing/purification or after relatively simple processing steps (e.g., centrifugation, precipitation, concentration or lyophilization). After the recombinant overexpression of the enzymes used, E. coli cells can alternatively also be used in the reaction directly without cell disintegration or, for example, after a freezing/thawing cycle. Suitable expression plasmids are known to a person skilled in the art and can often be purchased commercially.

“Functional variants” of cytochrome P450 can be fragments or mutational variants of cytochrome P450, wherein fragments of cytochrome P450 can also be referred to as “functional fragments”. “Functional variants” of cytochrome P450 are capable of catalyzing the same reaction as the protein from which they have been derived. Whether a variant is functional, i.e., whether it catalyzes the same reaction as the protein from which it is derived, can be determined by establishing that the variant catalyzes the same reaction. For this purpose, there are established methods in the prior art or, respectively, those that are described herein. The conversion rates of substrates by the functional variants according to the invention can deviate from the conversion rates of the cytochrome P450 from which they have been derived.

“Derivatives of 7-deoxysteroids” comprise compounds derived from 7-deoxysteroids and having a wide variety of modifications as defined above.

According to a preferred embodiment of the present invention, X₁, X₂, R₄ and R₅ are H and
R₁ und R₂ are independently H, OH, OR₈ or O, wherein
   R₈ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH₂(CH₃)₂, —C(O)C(CH), —C(O)Ph, —C(O)CH₂Ph,
R₃ is a C₁ to C₁₀ alkyl radical, a C₁ to C₁₀ alkylene radical, —CH(CH₃)((CH₂)₂CO₂R₉) or —CH(CH₃)((CH₂)₂CONHR₉), wherein
   R₉ is —CH₃, —CH₂COOH, —CH₂CH₃, —CH(CH₃)₂, —(CH₂)₂CH₃, —(C₂)₂SO₃H, C(CH₃)₃, —(CH₂)₃CH₃, —CH(CH₃)CH₂CH₃, —CH₂CH₂(CH₃)₂, an aryl group or an alkylaryl group.

According to a further preferred embodiment of the present invention, the aryl group is selected from the group consisting of a phenyl radical, a phenyl radical substituted with F, Cl, Br, NO₂ or CH₃ and a heteroaryl.

According to yet another preferred embodiment of the present invention, the alkylaryl group is selected from the group consisting of a benzyl group, a halogenated benzyl group, wherein the halogen is F, Cl or Br, and a benzyl group substituted with NO₂.

According to a preferred embodiment of the present invention, R₁ is OH, R₂ is O or OH, R₃ is CH(CH₃)((CH₂)₂CO₂R₅), R₄ is H, and R₅ is H.

According to another preferred embodiment of the present invention, the 7-deoxysteroid having the general formula (II) is selected from the group consisting of 3α,12α-dihydroxy-5β-cholane-24-acid, 3α,12β-dihydroxy-5β-cholane-24-acid, 3β,12α-dihydroxy-5β-cholane-24-acid, 3ß,12ß-dihydroxy-5ß-cholane-24-acid, 3ß-hydroxy-12-keto-5ß-cholane-24-acid, 3-keto, 12ß-hydroxy-5β-cholane-24-acid, 3-keto, 12α-hydroxy-5β-cholane-24-acid, 3α-hydroxy-5β-cholane-24-acid, 3-keto-5β-cholane-24-acid, 3β-hydroxy-5β-cholane-24-acid and esters of the respective acid.

The cytochrome P450 enzyme used, according to the invention, for the hydroxylation of 7-deoxysteroids and derivatives thereof having the general formula (II) to a steroid or a derivative thereof having the general formula (I) comprises an amino acid sequence which is at least 90%, more preferably at least 95%, more preferably at least 96%, more preferably at least 97%, more preferably at least 98%, more preferably at least 99%, in particular 100%, identical to the amino acid sequence SEQ ID No. 1 or 2.

SEQ ID No. 1:
MLTTAETTSIAYPFNTAEGLALSERYEEARNRTGLLRVRMPYGEPAWLVTRYADARLVLGDR
RFSRAEALHHDEPRQSEGRRDSGILTMDPPDHTRLRTLVAKAFTVHQVEKLRPWVRQLTHDL
LDDLEAAGPPADLVDRYALPIPVGVICAMLGVPQEDRPKFRVWSDAALSTSSLSAEQFARNT
DELRAYMAGLIEDHRRIPRDDIMTSLIEARDAGDRLSELELVDLCVGILVAGHETTATQIPN
FVLTLLEHPDQLRRLREDPALIQGAVEELLRFVPLGVGAAQARYATEDIEVGGTLVRSGEPV
LVAVGSANRDALRFDEPGVLNVARPTTQHLGFGHGVHHCLGAPLARLELQEALGALITRFPG
LRLAGDIEWKDRMLVRGPRVMPIGW
SEQ ID No. 2:
MPYGEPAWLVTRYADARLVLGDRRFSRAEALHHDEPRQSEGRRDSGILTMDPPDHTRLRTLV
AKAFTVHQVEKLRPWVRQLTHDLLDDLEAAGPPADLVDRYALPIPVGVICAMLGVPQEDRPK
FRVWSDAALSTSSLSAEQFARNTDELRAYMAGLIEDARRTPRDDIMTSLIEARDAGDRLSEL
ELVDLCVGILVAGHETTATQIFNFVLTLLEHPDQLRRLREDPALIQGAVEELLRFVPLGVGA
AQARYATEDIEVGGTLVRSGEPVLVAVGSANRDALRFDEPGVLNVARPTTQHLGFGHGVHHC
LGAPLARLELQEALGALITRFPGLRLAGDIEWKDRMLVRGPRVMPIGW

Amino acid sequences SEQ ID Nos. 1 and 2 are preferably encoded by nucleic acid sequences SEQ ID Nos. 3 and 4, with nucleic acid sequences SEQ ID Nos. 5 and 6 being optimized for expression in E. coli.

SEQ ID No. 3:
ATGTTGACCACAGCCGAGACGACATCCATCGCCTATCCCTTCAACACCGCCGAAGGGCTGGC
GCTCAGCGAGCGTTACGAAGAGGCCAGGAACCGCACCGGACTGCTCCGGGTGCGGATGCCCT
ACGGTGAGCCCGCCTGGCTGGTCACGCGGTACGCCGACGCCCGGCTGGTGCTCGGCGACCGG
CGCTTCAGCCGTGCGGAGGCGCTCCACCACGACGAGCCGCGGCAGTCCGAAGGCCGGCGCGA
CAGCGGCATCCTGACCATGGACCCGCCCGACCACACCCGGCTGCGCACCCTCGTCGCCAAGG
CGTTCACCGTCCACCAGGTGGAGAAACTCCGCCCCTGGGTACGCCAGTTGACCCATGACCTG
CTCGACGACCTCGAGGCCGCCGGGCCGCCCGCCGATCTGGTGGACCGCTACGCCCTGCCCAT
TCCGGTCGGCGTCATCTGCGCCATGCTCGGCGTCCCGCAGGAGGACCGGCCCAAGTTCCGGG
TCTGGAGCGACGCCGCGCTGTCCACCAGCTCGCTGAGCGCCGAGCAGTTCGCCCGTAACACC
GACGAGCTGCGCGCCTACATGGCCGGGCTGATCGAGGACCACCGCAGGACCCCGCGGGACGA
CATCATGACCTCGCTGATCGAGGCGCGGGACGCGGGCGACCGGCTGTCCGAGCTGGAACTCG
TCGATCTGTGCGTGGGCATCCTGGTGGCCGGGCACGAGACCACCGCCACCCAGATCCCCAAC
TTCGTGCTGACGCTGCTGGAGCACCCGGACCAGCTGCGCCGGCTGCGCGAGGACCCCGCCCT
GATCCAGGGCGCCGTCGAGGAGCTGCTGCGCTTCGTCCCGCTGGGCGTGGGCGCCGCCCAGG
CCCGTTACGCCACCGAGGACATCGAGGTGGGCGGCACGCTGGTGCGCAGCGGGGAGCCGGTG
CTGGTCGCCGTCGGCTCGGCCAACCGCGACGCGCTGCGCTTCGACGAACCGGGCGTGCTCAA
CGTCGCCCGCCCCACCACCCAGCACCTCGGCTTCGGCCACGGTGTGCACCACTGCCTGGGCG
CGCCCCTGGCCCGTCTGGAGCTCCAGGAGGCGCTCGGCGCGCTGATCACGCGCTTCCCGGGC
CTGCGGCTGGCCGGGGACATCGAGTGGAAGGACCGCATGCTGGTCCGCGGGCCCCGTGTCAT
GCCATCGGGTGGTGA
SEQ ID No. 4:
ATGCCCTACGGTGAGCCCGCCTGGCTGGTCACGCGGTACGCCGACGCCCGGCTGGTGCTCGG
CGACCGGCGCTTCAGCCGTGCGGAGGCGCTCCACCACGACGAGCCGCGGCAGTCCGAAGGCC
GGCGCGACAGCGGCATCCTGACCATGGACCCGCCCGACCACACCCGGCTGCGCACCCTCGTC
GCCAAGGCGTTCACCGTCCACCAGGTGGAGAAACTCCGCCCCTGGGTACGCCAGTTGACCCA
TGACCTGCTCGACGACCTCGAGGCCGCCGGGCCGCCCGCCGATCTGGTGGACCGCTACGCCC
TGCCCATTCCGGTCGGCGTCATCTGCGCCATGCTCGGCGTCCCGCAGGAGGACCGGCCCAAG
TTCCGGGTCTGGAGCGACGCCGCGCTGTCCACCAGCTCGCTGAGCGCCGAGCAGTTCGCCCG
TAACACCGACGAGCTGCGCGCCTACATGGCCGGGCTGATCGAGGACCACCGCAGGACCCCGC
GGGACGACATCATGACCTCGCTGATCGAGGCGCGGGACGCGGGCGACCGGCTGTCCGAGCTG
GAACTCGTCGATCTGTGCGTGGGCATCCTGGTGGCCGGGCACGAGACCACCGCCACCCAGAT
CCCCAACTTCGTGCTGACGCTGCTGGAGCACCCGGACCAGCTGCGCCGGCTGCGCGAGGACC
CCGCCTGATCCAGGGCGCCGTCGAGGAGCTGCTGCGCTTCTGTCCCGCTGGGCGTGGGCGCC
GCCCAGGCCCGTTACGCCACCGAGGACATCGAGGTGGGCGGCACGCTGGTGCGCAGCGGGGA
GCCGGTGCTGGTCGCCGTCGGCTCGGCCAACCGCGACGCGCTGCGCTTCGACGAACCGGGCG
TGCTCAACGTCGCCCGCCCCACCACCCAGCACCTCGGCTTCGGCCACGGTGTGCACCACTGC
CTGGGCGCGCCCCTGGCCCGTCTGGAGCTCCAGGAGGCGCTCGGCGCGCTGATCACGCGCTT
CCCGGGCCTGCGGCTGGCCGGGGACATCGAGTGGAAGGACCGCATGCTGGTCCGCGGGCCCC
GTGTCATGCCCATCGGGTGGTGA
SEQ ID No. 5:
ATGCTGACCACCGCAGAAACCACCAGTATTGCATATCCGTTTAATACCGCAGAAGGTCTGGC
ACTGAGCGAACGTTATGAAGAAGCACGTAATCGTACCGGTCTGCTGCGTGTTCGTATGCCGT
ATGGTGAACCGGCATGGCTGGTTACCCGTTATGCAGATGCCCGTCTGGTTCTGGGTGATCGT
CGTTTTAGCCGTGCCGAAGCACTGCATCACGATGAACCGCGTCAGAGCGAAGGTCGTCGTGA
TAGCGGTATTCTGACCATGGATCCGCCTGATCATACCCGTCTGCGTACCCTGGTTGCAAAAG
CATTTACCGTTCATCAGGTTGAAAAACTGCGTCCGTGGGTTCGCCAGCTGACCCATGATCTG
CTGGATGATCTGGAAGCAGCAGGTCCGCCTGCAGATCTGGTTGATCGTTATGCACTGCCGAT
TCCGGTTGGTGTTATTTGTGCAATGCTGGGTGTTCCGCAAGAAGATCGTCCTAAATTTCGTG
TTTGGAGTGATGCAGCACTGAGCACCAGCAGCCTGAGCGCAGAACAGTTTGCACGTAATACC
GATGAACTGCGTGCATATATGGCAGGTCTGATTGAAGATCATCGTCGTACACCGCGTGATGA
TATTATGACCAGCCTGATCGAAGCACGTGATGCCGGTGATCGCCTGAGTGAACTGGAACTGG
TGGATCTGTGTGTTGGTATTCTGGTTGCAGGTCATGAAACCACCGCAACCCAGATTCCGAAT
TTTGTTCTGACCCTGCTGGAACATCCGGATCAGCTGCGTCGTCTGCGTGAAGATCCGGCACT
GATTCAGGGTGCAGTTGAAGAACTGCTGCGTTTTGTTCCGCTGGGTGTGGGTGCAGCACAGG
CACGTTATGCAACCGAAGATATTGAAGTTGGTGGCACCCTGGTTCGTAGTGGCGAACCGGTG
CTGGTTGCCGTTGGTAGCGCAAACCGTGATGCACTGCGCTTTGATGAACCGGGTGTTCTGAA
TGTTGCACGTCCGACCACACAGCATCTGGGTTTTGGTCATGGTGTTCATCATTGTCTGGGTG
CACCGCTGGCACGTCTGGAACTGCAAGAAGCACTGGGAGCACTGATTACCCGTTTTCCGGGT
CTGCGTCTGGCAGGCGATATTGAATGGAAAGATCGTATGCTGGTTCGTGGTCCGCGTGTTAT
GCCGATTGGTTGGTAA
SEQ ID No. 6:
ATGGTGAACCGGCATGGCTGGTTACCCGTTATGCAGATGCCCGTCTGGTTCTGGGTGATCGT
CGTTTTAGCCGTGCCGAAGCACTGCATCACGATGAACCGCGTCAGAGCGAAGGTCGTCGTGA
TAGCGGTATTCTGACCATGGATCCGCCTGATCATACCCGTCTGCGTACCCTGGTTGCAAAAG
CATTTACCGTTCATCAGGTTGAAAAACTGCGTCCGTGGGTTCGCCAGCTGACCCATGATCTG
CTGGATGATCTGGAAGCAGCAGGTCCGCCTGCAGATCTGGTTGATCGTTATGCACTGCCGAT
TCCGGTTGGTGTTATTTGTGCAATGCTGGGTGTTCCGCAAGAAGATCGTCCTAAATTTCGTG
TTTGGAGTGATGCAGCACTGAGCACCAGCAGCCTGAGCGCAGAACAGTTTGCACGTAATACC
GATGAACTGCGTGCATATATGGCAGGTCTGATTGAAGATCATCGTCGTACACCGCGTGATGA
TATTATGACCAGCCTGATCGAAGCACGTGATGCCGGTGATCGCCTGAGTGAACTGGAACTGG
TGGATCTGTGTGTTGGTATTCTGGTTGCAGGTCATGAAACCACCGCAACCCAGATTCCGAAT
TTTGTTCTGACCCTGCTGGAACATCCGGATCAGCTGCGTCGTCTGCGTGAAGATCCGGCACT
GATTCAGGGTGCAGTTGAAGAACTGCTGCGTTTTGTTCCGCTGGGTGTGGGTGCAGCACAGG
CACGTTATGCAACCGAAGATATTGAAGTTGGTGGCACCCTGGTTCGTAGTGGCGAACCGGTG
CTGGTTGCCGTTGGTAGCGCAAACCGTGATGCACTGCGCTTTGATGAACCGGGTGTTCTGAA
TGTTGCACGTCCGACCACACAGCATCTGGGTTTTGGTCATGGTGTTCATCATTGTCTGGGTG
CACCGCTGGCACGTCTGGAACTGCAAGAAGCACTGGGAGCACTGATTACCCGTTTTCCGGGT
CTGCGTCTGGCAGGCGATATTGAATGGAAAGATCGTATGCTGGTTCGTGGTCCGCGTGTTAT
GCCGATTGGTTGGTAA

“Identical” as used herein means that two or more amino acid sequences, when superimposed on one another, may have a certain “identity” (matching amino acid residues at identical positions) to one another. “Identity” is defined in this invention as the percentage of amino acids of eligible amino acid sequences that are identical to the amino acids of the starting sequence, namely after the alignment of the two sequences and the introduction of gaps, if necessary, in order to achieve the maximum percentual sequence identity as generated by the “protein BLAST” program (blastp; Altschul et al., J. Mol. Biol. (1997) 215:403-410; http://blast.ncbi.nlm.nih.gov/Blast.cgi; commonly referred to herein as “BLAST”), with all variable parameters set to default values. Herein, the algorithm “blastp (protein-protein-BLAST)” is used with the following parameters: “expect threshold”: 0.05; “word size”: 6; matrix: BLOSUM62; “gap costs”: “Existence” 11, “Extension” 1; conditional compositional score matrix adjustment; no filter and no mask. A percentage (%) value for the amino acid sequence identity is determined by the number of matching identical nucleotides divided by the sequence length for which the identity in percent is recorded.

Using the method according to the invention, 7-deoxysteroids or, respectively, derivatives thereof having the general formula (II) can be converted with cytochrome P450 or a functional variant thereof to steroids or, respectively, derivatives thereof having the general formula (I), with the cytochrome P450 enzyme comprising an amino acid sequence which is at least 90%, in particular 100%, identical to the amino acid sequence SEQ ID No. 1 or 2. This conversion takes place in the presence of redox partners or a redox partner system which is able to provide electrons for the hydroxylation reaction.

According to a preferred embodiment of the present invention, the at least one redox partner system comprises
(i) ferredoxin, ferredoxin reductase and NAD(P)H;
(ii) cytochrome P450 reductase and NAD(P)H; or
(iii) NAD(P)H.

The redox partner system used according to the invention can comprise ferredoxin, ferredoxin reductase and NAD(P)H; cytochrome P450 reductase and NAD(P)H; or NAD(P)H alone, with a redox partner system comprising ferredoxin, ferredoxin reductase and NAD(P)H being particularly preferred.

In order to carry out the redox reaction of cytochrome P450 according to the invention or, respectively, the functional variants thereof, it is therefore advantageous to use at least the redox cofactors NAD+/NADH and/or NADP+/NADPH in the method according to the invention. In this context, NAD+ designates the oxidized form and NADH designates the reduced form of nicotinamide adenine dinucleotide, whereas NADP+ designates the oxidized form and NADPH designates the reduced form of nicotinamide adenine dinucleotide phosphate.

The concentration of the redox cofactors NAD(P)+ and/or NAD(P)H in a reaction mixture is preferably between 0.001 mM and 10 mM, more preferably between 0.05 mM and 1 mM.

Particularly preferably, ferredoxins are used as redox partners, which can be regenerated in the presence of NAD(P)+ and at least one ferredoxin reductase. According to a preferred embodiment of the present invention, the at least one ferredoxin is selected from the group consisting of adrenodoxins, putidaredoxins and flavodoxins, wherein, optionally, combinations thereof can be used as well.

A possible pair of redox partners preferably comprises putidaredoxin and putidaredoxin reductase from Pseudomonas putida. Moreover, a person skilled in the art is able to identify further ferredoxin proteins and ferredoxin reductases which are potential redox partners for the cytochrome P450 according to the invention. Suitability as a redox partner can be verified in a functional assay, as described, for example, in Examples 3 to 5. The putidaredoxin used in these examples and/or the putidaredoxin reductase used therein can be replaced by possible alternative proteins or enzymes, respectively. If sufficient formation of the desired product (e.g., ursocholic acid) is observed, the tested redox partners can be regarded as functional alternatives to putidaredoxin and/or putidaredoxin reductase.

According to a particularly preferred embodiment of the present invention, the ferredoxin used in the method according to the invention comprises an amino acid sequence which is at least 80%, preferably at least 85%, more preferably at least 90%, more preferably at least 95%, more preferably at least 96%, more preferably at least 97%, more preferably at least 98%, more preferably at least 99%, in particular 100%, identical to the amino acid sequence SEQ ID No. 7, wherein X is a methionine residue or is not an amino acid.

SEQ ID No. 7:
XSKVVYVSHDGTRRELDVADGVSLMQAAVSNGIYDIVGDCGGSASCATC
HVYVNEAFTDKVPAANEREIGMLECVTAELKPNSRLCCQIIMTPELDGI
VVDVPDRQW

According to a preferred embodiment of the present invention, the at least one ferredoxin reductase is selected from the group of flavodoxin reductases and putidaredoxin reductase.

The ferredoxin oxidized in the course of the hydroxylation reaction according to the invention can be reduced with the aid of a ferredoxin reductase and NAD(P)H. As a result, reduced ferredoxin is again provided or, respectively, regenerated while consuming NAD(P)H for a further hydroxylation reaction of the substrate according to the invention. The ferredoxin reductase can be a flavodoxin reductase and/or a putidaredoxin reductase.

According to a further preferred embodiment of the present invention, the ferredoxin reductase used in the method according to the invention comprises an amino acid sequence which is at least 80%, preferably at least 85%, more preferably at least 90%, more preferably at least 95%, more preferably at least 96%, more preferably at least 97%, more preferably at least 98%, more preferably at least 99%, in particular 100%, identical to the amino acid sequence SEQ ID No. 8.

SEQ ID No. 8:
MNANDNVVIVGTGLAGVEVAFGLRASGWEGNIRLVGDATVIPHHLPPLSKAYLAGKATAE
SLYLRTPDAYAAQNIQLLGGTQVTAINRDRQQVILSDGRALDYDRLVLATGGRPRPLPVA
SGAVGKANNFRYLRTLEDAECIRRQLIADNRLVVIGGGYIGLEVAATAIKANMHVTLLDT
AARVLERVTAPPVSAFYEHLHREAGVDIRTGTQVCGFEMSTDQQKVTAVLCEDGTRLPAD
LVIAGIGLIPNCELASAAGLQVDNGIVINEHMQTSDPLIMAVGDCARFHSQLYDRWVRIE
SVPNALEQARKIAAILCGKVPRDEAAPWFWSDQYEIGLKMVGLSEGYDRIIVRGSLAQPD
FSVFYLQGDRVLAVDTVNRPVEFNQSKQIITDRLPVEPNLLGDESVPLKEIIAAAKAELS
SA

Amino acid sequences SEQ ID Nos. 7 and 8 are preferably encoded by nucleic acid sequences SEQ ID Nos. 9 and 10, respectively, with nucleic acid sequences SEQ ID Nos. 11 and 12 being optimized for expression in E. coli.

SEQ ID No. 9:
(ATG)_{0 or 1}
TCTAAAGTAGTGTATGTGTCACATGATGGAACGCGTCGCGAACTGGATGTGGCGGATGGC
GTCAGCCTGATGCAGGCTGCAGTCTCCAATGGTATCTACGATATTGTCGGTGATTGTGGC
GGCAGCGCCAGCTGTGCCACCTGCCATGTCTATGTGAACGAAGCGTTCACGGACAAGGTG
CCCGCCGCCAACGAGCGGGAAATCGGCATGCTGGAGTGCGTCACGGCCGAACTGAAGCCG
AACAGCAGGCTCTGCTGCCAGATCATCATGACGCCCGAGCTGGATGGCATCGTGGTCGAT
GTTCCCGATAGGCAATGGTAA
SEQ ID No. 10:
ATGAACGCAAACGACAACGTGGTCATCGTCGGTACCGGACTGGCTGGCGTTGAGGTCGCC
TTCGGCCTGCGCGCAAGCGGCTGGGAAGGCAATATCCGGTTGGTGGGGGATGCGACGGTA
ATTCCCCATCACCTACCACCGCTATCCAAAGCTTACTTGGCCGGCAAAGCCACAGCGGAA
ACACAGGTAACGGCTATCAACCGCGACCGACAGCAAGTAATCCTATCGGATGGCCGGGCA
CTGGATTACGACCGGCTGGTATTGGCTACCGGAGGGCGTCCAAGACCCCTACCGGTGGCC
AGTGGCGCAGTTGGAAAGGCGAACAACTTTCGATACCTGCGCACACTCGAGGACGCCGAG
TGCATTCGCCGGCAGCTGATTGCGGATAACCGTCTGGTGGTGATTGGTGGCGGCTACATT
GGCCTTGAAGTGGCTGCCACCGCCATCAAGGCGAACATGCACGTCACCCTGCTTGATACG
GCAGCCCGGGTTCTGGAGCGGGTTACCGCCCCGCCGGTATCGGCCTTTTACGAGCACCTA
CACCGCGAAGCCGGCGTTGACATACGAACCGGCACGCAGGTGTGCGGGTTCGAGATGTCG
ACCGACCAACAGAAGGTTACTGCCGTCCTCTGCGAGGACGGCACAAGGCTGCCAGCGGAT
CTGGTAATCGCCGGGATTGGCCTGATACCAAACTGCGAGTTGGCCAGTGCGGCCGGCCTG
CAGGTTGATAACGGCATCGTGATCAACGAACACATGCAGACCTCTGATCCCTTGATCATG
GCCGTCGGCGACTGTGCCCGATTTCACAGTCAGCTCTATGACCGCTGGGTGCGTATCGAA
TCGGTGCCCAATGCCTTGGAGCAGGCACGAAAGATCGCCGCCATCCTCTGTGGCAAGGTG
CCACGCGATGAGGCGGCGCCCTGGTTCTGGTCCGATCAGTATGAGATCGGATTGAAGATG
GTCGGACTGTCCGAAGGGTACGACCGGATCATTGTCCGCGGCTCTTTGGCGCAACCCGAC
TTCAGCGTTTTCTACCTGCAGGGAGACCGGGTATTGGCGGTCGATACAGTGAACCGTCCA
GTGGAGTTCAACCAGTCAAAACAAATAATCACGGATCGTTTGCCGGTTGAACCAAACCTA
CTCGGTGACGAAAGCGTGCCGTTAAAGGAAATCATCGCCGCCGCCAAAGCTGAACTGAGT
AGTGCCTGA
SEQ ID No. 11:
(ATG)_{0 or 1}
ATGAGCAAAGTGGTCTATGTGTCGCATGATGGAACACGCCGTGAGTTAGACGTCGCTGAT
GGTGTATCCCTGATGCAAGCAGCGGTTAGCAATGGCATTTACGACATCGTTGGCGATTGT
GGTGGTAGTGCGTCATGTGCAACGTGTCACGTGTATGTTAACGAAGCGTTTACCGATAAG
GTGCCTGCTGCCAATGAACGCGAGATTGGCATGCTGGAATGCGTAACTGCCGAACTCAAA
CCGAACTCTCGCCTGTGCTGCCAGATCATCATGACCCCGGAATTGGACGGGATTGTCGTT
GATGTGCCAGATCGTCAGTGGTAA
SEQ ID No. 12:
ATGAACGCCAATGATAATGTTGTTATTGTTGGCACCGGTCTGGCAGGCGTTGAAGTTGCA
TTTGGTCTGCGTGCAAGCGGTTGGGAAGGTAATATTCGTCTGGTTGGTGATGCAACCGTT
ATTCCGCATCATCTGCCTCCGCTGAGCAAAGCATATCTGGCAGGTAAAGCAACCGCAGAA
AGCCTGTATCTGCGTACACCGGATGCCTATGCAGCACAGAATATTCAGCTGCTGGGTGGT
ACACAGGTTACCGCAATTAATCGTGATCGTCAGCAGGTTATTCTGAGTGATGGTCGTGCA
CTGGATTATGATCGTCTGGTGCTGGCAACCGGTGGTCGTCCGCGTCCGCTGCCGGTTGCA
AGTGGTGCAGTTGGTAAAGCCAATAACTTTCGTTATCTGCGCACCCTGGAAGATGCAGAA
TGTATTCGTCGTCAGCTGATTGCAGATAATCGCCTGGTTGTGATTGGTGGTGGTTATATT
GGTCTGGAAGTTGCAGCAACCGCCATTAAAGCAAATATGCATGTTACCCTGCTGGATACC
GCACCACGTGTTCTGGAACGTGTTACCGCACCGCCTGTTAGCGCCTTTTATGAACATCTG
CATCGTGAAGCCGGTGTTGATATTCGTACCGGCACCCAGGTTTGTGGTTTTGAAATGAGC
ACCGATCAGCAGAAAGTTACCGCAGTTCTGTGTGAAGATGGCACCCGTCTGCCTGCAGAT
CTGGTTATTGCAGGTATTGGCCTGATTCCGAATTGTGAACTGGCAAGCGCAGCAGGTCTG
CAGGTTGATAATGGTATTGTTATTAACGAACACATGCAGACCAGCGATCCGCTGATTATG
GCAGTTGGTGATTGTGCACGTTTTCATAGCCAGCTGTATGATCGTTGGGTTCGTATTGAA
AGCGTTCCGAATGCACTGGAACAGGCACGTAAAATTGCAGCAATTCTGTGTGGTAAAGTT
CCGCGTGATGAAGCAGCACCGTGGTTTTGGAGCGATCAGTATGAAATTGGTCTGAAAATG
GTTGGTCTGAGCGAAGGTTATGATCGCATTATTGTTCGTGGTAGCCTGGCACAGCCGGAT
TTTTCAGTTTTTTATCTGCAGGGTGATCGTGTGCTGGCAGTTGATACCGTTAATCGTCCG
GTTGAATTTAACCAGAGCAAACAAATTATCACCGATCGTCTGCCGGTGGAACCGAATCTG
CTGGGAGATGAAAGCGTGCCGCTGAAAGAAATTATTGCAGCAGCAAAAGCAGAACTGAGC
AGCGCATA

The expression of the cytochrome P450 according to the invention and any ferredoxins and ferredoxin reductases in bacteria, in particular in E. coli, is particularly advantageous when nucleic acids with the nucleic acid sequences SEQ ID No. 5, SEQ ID No. 6, SEQ ID No. 11 and/or SEQ ID No. 12 are used. Further aspects of the present invention therefore relate to a nucleic acid (DNA and/or RNA) with a nucleic acid sequence selected from the group consisting of SEQ ID No. 5, SEQ ID No. 6, SEQ ID No. 11 and SEQ ID No. 12 and vectors and/or cells, in particular E. coli cells, comprising at least one of those sequences.

It has been shown that it is advantageous if the above-mentioned ferredoxins and ferredoxin reductases are expressed (co-expressed) together with cytochrome P450 in a production strain (e.g., an E. coli strain). The ferredoxins, ferredoxin reductases and the cytochrome P450 can also be expressed separately from one another. It is also advantageous to co-express ferredoxin and cytochrome P450 or ferredoxin reductase and cytochrome P450. Through the co-expression of the three proteins or, respectively, enzymes, ideally under the same promoter, an ideal balance between the enzymes can be established, which has a particularly advantageous effect on the enzymatic conversion of a substrate.

According to a preferred embodiment of the present invention, the at least one oxidoreductase is selected from the group consisting of oxidoreductase (EC: 1.1.1), aldehyde dehydrogenase (EC: 1.2.1), amino acid dehydrogenase (EC: 1.4.1), flavin reductase (EC: 1.5.1), transhydrogenase (EC: 1.6.1), nitrite reductase (EC: 1.7.1) and phosphonate dehydrogenase (EC: 1.20.1), preferably selected from the group consisting of alcohol dehydrogenase, hydroxysteroid dehydrogenase, phosphite dehydrogenase and sugar dehydrogenase.

In order to regenerate the redox partner system used in the method according to the invention, in particular the cofactor used in the process (NADH/NAD⁺ and/or NADPH/NADP⁺), during the conversion of the steroid having the general formula (I) to a 7-deoxysteroid having the general formula (II), so that the conversion reaction is pushed towards the product, it is advantageous to add a system for regenerating the redox partner system, advantageously oxidoreductases, to the reaction mixture. Oxidoreductases convert substrates by reduction and oxidation, wherein, in the course of those reactions, NADH is oxidized to NAD⁺ and NADPH is oxidized to NADP⁺ or, respectively, NAD⁺ is reduced to NADH and NADP⁺ is reduced to NADPH. Therefore, the system for regenerating the redox partner system preferably comprises at least one oxidoreductase and at least one substrate of the at least one oxidoreductase.

The oxidoreductase used in the method according to the invention is preferably an alcohol and/or sugar dehydrogenase.

According to a further preferred embodiment of the present invention, the oxidoreductase is selected from the group consisting of glucose dehydrogenase, glucose-6-phosphate dehydrogenase, arabinose dehydrogenase, xylose dehydrogenase, sorbitol dehydrogenase, xylitol dehydrogenase, 12α-hydroxysteroid dehydrogenase, 7α-hydroxysteroid dehydrogenase, 20α-hydroxysteroid dehydrogenase, 170-hydroxysteroid dehydrogenase, 17α-hydroxysteroid dehydrogenase, 3α-hydroxysteroid dehydrogenase, 3β-hydroxy-delta5 dehydrogenase, 11β-hydroxysteroid dehydrogenase and formate dehydrogenase.

The use of one or several of the above-mentioned oxidoreductases is particularly advantageous for recycling the cofactors used in the conversion reaction.

It has been shown that it is particularly advantageous to add arabinose dehydrogenase, sorbitol dehydrogenase and/or xylitol dehydrogenase to the reaction mixture in order to achieve a high conversion of the substrate into the product.

The reaction mixture can comprise at least one oxidoreductase and one hydroxylase. It is particularly advantageous to add a combination of two or three or more oxidoreductases to the reaction mixture, with a combination of a 12α-hydroxysteroid dehydrogenase and a 7α-hydroxysteroid dehydrogenase or, respectively, a 12α-hydroxysteroid dehydrogenase, a 7α-hydroxysteroid dehydrogenase and an NAD(P)H oxidase or, respectively, an NADH-dependent alcohol dehydrogenase and a hydroxylase or, respectively, an NADPH-dependent alcohol dehydrogenase and a hydroxylase being particularly well suited for the substrate mixtures, for example, for the simultaneous oxidation and hydroxylation of naturally occurring mixtures of cholic acids.

For catalyzing the oxidation or, respectively, reduction reactions of the cofactors in the reaction mixture of the method according to the invention, it is necessary to provide at least one substrate for the oxidoreductases present therein. Therefore, the reaction mixture comprises at least one substrate of the at least one oxidoreductase selected from the group consisting of arabinose, xylose, glucose, sorbitol, xylitol, cholane-24-acid, 3α,12α-dihydroxycholane-24-acid-2,3-butanediol, acetoin, 2-propanol, glutamates, ethanol, phosphonates, phosphites, nitrites, 4-methyl-2-pentanol, 2-butanol, 2-octanol, cyclohexanol, ethanediol, 1,2-propanediol, 1-propanol, 1-butanol, 3-hydroxybutanoate and formate. According to a preferred embodiment of the present invention, the method according to the invention is performed at a temperature of from 10° C. to 40° C., preferably from 15° C. to 38° C., more preferably from 20° C. to 30° C., more preferably from 22° C. to 26° C. It has been shown according to the invention that the enzyme activity of cytochrome P450 for the reaction according to the invention is particularly high in this area.

According to a further preferred embodiment of the present invention, the method according to the invention is performed at a pH of from 6.5 to 8.5, preferably from 7 to 8, more preferably from 7.2 to 7.8. At this pH value, the enzyme activity of cytochrome P450 is highest so as to allow an appropriate conversion of the substrate.

The hydroxylation of deoxysteroids or, respectively, deoxysteroid derivatives can be carried out regioselectively at position 7 of the steroid backbone. In this way, in particular, a 7beta-hydroxyl group can be introduced stereoselectively so that, for example, ursocholic acid and/or ursocholic acid derivatives can be produced.

In a preferred embodiment, the method according to the invention is performed in the presence of at least one organic solvent. Preferably, a single organic solvent is used so that a single-phase system is provided. It is also possible to use a mixture of two or more organic solvents which, according to the invention, are miscible with each other so that a single-phase system is provided. The organic solvent can be protic or aprotic, with aprotic solvents being preferred.

Surprisingly, it has been found that the presence of an organic solvent, in particular an aprotic organic solvent, can significantly increase the conversion of the 7-deoxysteroid of general formula (II) to the steroid of general formula (I) according to the method according to the invention. In addition, the implementation of the method according to the invention in the presence of the organic solvent surprisingly allows the redox partner system to be regenerated.

Alcohols, ethers, esters, glycols, ketones, amides, sulfoxides, organic acids, cycloalkanes, aromatics and chlorinated hydrocarbons can, for example, be used as organic solvents. Examples of suitable organic solvents are methanol, ethanol, isopropanol, 2-butanol, 4-methyl-2-pentanol (methyl isobutyl alcohol, MIBA), diethyl ether (Et₂O), diisopropyl ether (iPr₂O), dioxane, tetrahydrofuran (THF), 2-methyltetrahydrofuran (Me-THF), ethyl acetate, ethylene glycol, methyl isobutyl ketone (MIBK), 2-butanone, acetone, dimethyl sulfoxide (DMSO), dimethylformamide (DMF), dimethylacetamide (DMA), cyclohexane, toluene, trichloromethane (CHCl₃), dichloromethane (CH₂Cl₂), hexane or mixtures thereof. Suitable mixtures are, for example, mixtures of hexane and ethyl acetate or isopropanol, as well as mixtures of trichloromethane and phenol. The present invention is not limited to the above exemplary list of solvents.

The organic solvent is preferably an aprotic organic solvent, particularly preferably a solvent selected from the group consisting of dimethylformamide (DMF), dimethyl sulfoxide (DMSO) and dimethylacetamide (DMA).

According to the invention, the amount of the organic solvent is chosen such that the compound of general formula (II) is completely dissolved and the enzyme activity is preserved. Preferably, the compound of formula (II), e.g., lithocholic acid, is dissolved in the organic solvent up to the limit of solubility. In a preferred embodiment, the substrate of the enzyme is placed in the organic solvent.

In the method according to the present invention, the isolation of the product can be effected in different ways. For example, the product can be extracted from the reaction mixture by a suitable organic solvent. Depending on the substrate, such solvents are described in the literature. According to the present invention, cholic acids and their derivatives can be isolated from reaction mixtures, for example, with ethyl acetate, optionally after acidification of the reaction mixture, e.g., with HCl. A method in which bile acids are present in the form of a salt, e.g., a sodium salt, in an aqueous solution constitutes a special case.

In this case, a precipitation of the product can be effected by acidifying the reaction mixture. For this purpose, for example, HCl or dilute HCl can be added to the reaction mixture in a sufficient amount. If a pH value of, for example, 1 to 4, preferably 2 to 3, is achieved in the process, the product predominantly exists in the form of a suspension. The product can then be removed from the reaction mixture by common methods such as, e.g., filtration or centrifugation. Chromatographic methods, such as, e.g., affinity chromatography or ion-exchange chromatography, are another alternative that can be used for product isolation, for example. Furthermore, it is possible, for example, to obtain product by evaporating the reaction solvent.

Alternatively, in a method according to the present invention, the product(s) may also remain in the reaction mixture after the reaction, e.g., in order to carry out even more reactions and optionally isolate an end product upon completion of those reactions. It is also conceivable that the substrate(s) for the method according to the present invention is/are produced in the same reaction batch by previous reactions or reactions taking place in parallel.

A further aspect of the present invention relates to a nucleic acid construct comprising a nucleic acid molecule coding for a cytochrome P450 enzyme as defined above to which 3′ end and/or 5′ end at least one nucleic acid molecule coding for a polypeptide selected from the group consisting of a ferredoxin, a ferredoxin reductase and an oxidoreductase is bound directly or via a spacer.

The nucleic acid constructs according to the invention are particularly suitable for the production of a steroid as initially defined. By expressing the cytochrome P450 enzyme and at least one protein selected from the group consisting of ferredoxins, ferredoxin reductases and oxidoreductases, starting from a nucleic acid construct, it becomes possible to produce these enzymes or, respectively, proteins in an amount which is necessary for an efficient implementation of the method according to the invention. It is particularly advantageous if all of these proteins are expressed under the control of the same promoter on the nucleic acid construct according to the invention. A further aspect of the present invention is a vector comprising a nucleic acid construct according to the present invention.

The nucleic acid molecule coding for a cytochrome P450 enzyme can be bound to further nucleic acid molecules coding for enzymes or, respectively, proteins, which can be used in the method according to the invention, either directly or via a spacer or, respectively, a spacer sequence. The advantage of such a construct is that such a construct allows to express the enzymes and proteins used in the method according to the invention, in particular cytochrome P450 and ferredoxin and/or ferredoxin reductase, in a comparable amount.

A “spacer” or, respectively, a “spacer sequence” as used herein is a nucleic acid sequence which has neither a stop codon nor any other functional motif A spacer or, respectively, a spacer sequence serves as a distance holder between two ORFs in order to improve the transcription of these ORFs, if necessary.

According to a preferred embodiment of the present invention, the cytochrome P450 enzyme is encoded by a nucleic acid which is at least 90%, in particular 100%, identical to the nucleic acid sequence SEQ ID No. 3, SEQ ID No. 4, SEQ ID No. 5 or SEQ ID No. 6.

According to a further preferred embodiment of the present invention, the ferredoxin comprises the amino acid sequence SEQ ID No. 7.

According to a particularly preferred embodiment of the present invention, the ferredoxin reductase is an adrenodoxin reductase, preferably a putidaredoxin reductase.

The ferredoxin reductase preferably comprises the amino acid sequence SEQ ID No. 8.

Another aspect of the present invention relates to a vector comprising a nucleic acid construct according to the present invention.

The vector according to the invention can be a cloning or expression vector and, depending on the organism into which it is introduced, can have appropriate sections in order to enable, for example, the transcription of an ORF.

Yet another aspect of the present invention relates to a host cell comprising a nucleic acid construct according to the present invention.

The host cell according to the invention can be used for cloning or for expressing the ORFs that have been introduced recombinantly and are located on a nucleic acid construct.

The lysate of such a host cell can be used in the method according to the invention, provided that the host cell has intracellularly or extracellularly expressed at least one of the enzymes or, respectively, proteins required in the method according to the invention. The 7-deoxy steroid or a derivative thereof having the general formula (II) is thereby preferably brought into contact with a cell suspension or cells in a culture supernatant and/or a lysate of a host cell according to the present invention. Thus, according to a preferred embodiment of the present invention, 7-deoxy steroid or a derivative thereof having the general formula (II) is brought into contact with at least one culture supernatant and/or a lysate of at least one host cell capable of expressing at least one ferredoxin, at least one ferredoxin reductase and/or at least one oxidoreductase.

### EXAMPLES

The present invention is explained in further detail using the following examples, without, however, being restricted thereto.

### Example 1: Test of Bacterial Strains

The following bacterial strains were obtained from the German Strain Collection of Microorganisms and Cell Cultures (DSMZ [Deutsche Stammsammlung für Mikroorganismen und Zellkulturen]): Saccharothrix longispora (DSM-43749), Catellatospora citrae (DSM-44097), Streptomyces hygroscopicus subsp. hygroscopicus (DSM-40578) and Asanoa ferruginea (DSM-44099). The strains were cultivated under standard conditions as recommended by DSMZ. As soon as the growth of the cultures had led to visible turbidity, deoxycholic acid (0.5 mM) was added, and it was cultured further for up to 72 h. After a centrifugation step, supernatants of the cultures were extracted with ethyl acetate and analyzed by HPLC and GC/MS. In the HPLC chromatogram of the reaction with Streptomyces hygroscopicus, a peak was noted the retention time of which corresponds to that of ursocholic acid. The GC/MS analysis indicated that the potential ursocholic acid peak originates from a bile acid with 3 hydroxyl groups. The examination of the other strains gave no indication of 7-hydroxylated products of deoxycholic acid.

### Example 2: Genome Sequencing and Annotation of the P450 Genes

Upon cultivation of Streptomyces hygroscopicus subsp. hygroscopicus (DSM-40578) according to the DSMZ regulation, the genomic DNA of the strain was isolated (Kieser et al. (2000), Practical Streptomyces genetics (Norwich: John Innes Foundation)). The genome was sequenced using Illumina MiSeq, and an assembly based on the known genome of Streptomyces rapamycinicus was conducted (Microsynth GmbH, Switzerland). 42 P450 genes could be identified by homology comparisons.

### Example 3: Cloning of Expression System

Using the restriction enzyme XhoI, the following construct comprising coding regions for putidaredoxin reductase (PtR) and putidaredoxin (Ptx) was cloned into plasmid pJ411 (DNA 2.0).

Synthetic DNA (Life Technologies): 5′, XhoI interface, HindIII interface, approx. 50 bp spacer DNA, ribosome binding site (rbs), ORF (open reading frame) putidaredoxin reductase (PtR), approx. 50 bp spacer DNA, rbs, ORF putidaredoxin (Ptx), XhoI interface, 3′.

The result of the cloning step was checked by means of restriction enzyme digestion and DNA sequencing.

Subsequently, using the restriction enzymes NdeI and HindIII, one ORF each coding for the P450 enzymes identified in Example 2 was cloned into the above-mentioned synthetic DNA or plasmid, respectively (Life Technologies). The result was again verified by means of restriction enzyme digestion and DNA sequencing. The expression vector used in this example and the redox partners used constitute only one way of expressing the cytochrome P450 enzymes according to the invention, which way has been chosen as an example.

The expression plasmids produced with the identified P450 candidates (see example 2) can be used for jointly expressing the respective P450 proteins together with putidaredoxin reductase and putidaredoxin. The 3 ORFs of the respective expression plasmids are expressed under the control of a T7 promoter on a common mRNA, but as separate polypeptides.

### Example 4: Expression of P450/Ptx/PtR

After the genome sequencing of Streptomyces hygroscopicus subsp. hygroscopicus, there were 42 P450 sequences that came into consideration as candidates for a possible deoxycholic acid-7-hydroxylase. To identify the enzyme looked for, ORFs of the candidates were cloned into the expression system described in example 3 and into a pJ411 (DNA 2.0) expression vector without coding regions for putidaredoxin reductase (PtR) and putidaredoxin (Ptx). The following protocol was used for the expression.

TB-P450 expression medium:
Terrific broth (TB) medium
+50 μg/ml kanamycin
+0.5 mM 5-aminolevulinic acid (from 100× parent solution)
+1 mM thiamine (from 100× parent solution)
+1 mM MgCl₂+2.5 mM ammonium sulfate+50 μM FeCl₃ (from 100× parent solution)
+0.5 mM IPTG (from 1 M parent solution)
(the additives were each 0.2 μm sterile filtered)
   P450 lysis buffer:
100 mM Tris pH 7.5
20% (v/v) glycerin
1 mg/ml lysozyme

The constructs of the P450 candidates, which were to be tested, were transformed into the E. coli expression strain BL21 (DE3). Overnight cultures were inoculated from single colonies (LB (lysogeny broth)+kanamycin). The next day, 1:100 expression cultures were inoculated therewith (150 ml TB (terrific broth)—P450 expression medium) and were initially shaken at 37° C. in baffled flasks (1 L) for 3 h. Subsequently, the temperature was lowered to 24° C., and it was shaken for another 22 h. The cultures were harvested by centrifugation at 5000 g for 10 min, washed 1× with 0.9% (w/v) NaCl, and pellets were frozen at −80° C. The cell pellets were thawed, weighed and resuspended with an equivalent amount of P450 lysis buffer, incubated on ice for 1 h and then digested using a sonifier. Upon centrifugation (30 min, 21000 g), the supernatants were used for test reactions.

### Example 5: Testing of P450 Candidates for DA Hydroxylation

Reaction mixture:
10-80 μl 100 mM NADH (redox cofactor)
250 μl 1 M Tris-HCl pH 7.5
17.5 μl glycerin (50%)
100 μl 50 mM deoxycholic acid solution pH 8.5 (final 10 mM)
50 μl E. coli lysate P450/PtR/Ptx (see Example 4)
17.5-87.5 μl dH₂O

The reactions were set up in 1.5 ml screw-top bottles and sealed with lids with aluminium foil. The foil was punctured in several places. It was gently shaken at 24° C. for 18 h. 200 μl of the reaction batch was diluted with 600 μl acetonitrile/5 μl H₃βO₄ (50%) and incubated at 55° C. for 15 minutes. Subsequently, the samples were centrifuged at 20817 rcf for 5 minutes and analyzed using HPLC/DAD (e.g., Agilent 1200 series;
column: Merck Purospher STAR RP-18e 125×4 mm, 5 μm;
flow rate: 1.5 ml/min, gradient H₂O+H₃βO₄ (pH=2.6)/acetonitrile). One of the examined candidates (“P450_c866”) was able to hydroxylate deoxycholic acid to ursocholic acid. The deoxycholic acid used was converted in the process (see the following table). The identity of the product ursocholic acid was verified by GC/MS analysis and by 2D NMR.

| Redox cofactor [μl] | ursocholic acid [μg/ml] | conversion [%] |
|---|---|---|
| 10 | 30 | 3.4 |
| 30 | 48 | 5.3 |
| 50 | 57 | 6.2 |
| 80 | 110 | 11.6 |

In this example, a redox cofactor (NADH) is oxidized by the P450/Ptx/PtR reaction.

### Example 6: Testing of P450 Candidates for DA Hydroxylation with Cofactor Recycling of Arabinose Dehydrogenase

Reaction mixture:
65 μl 100 mM NADH (final 0.5 mM)
1 ml 1 M Tris-HCl pH 7.5+20% (v/v) glycerin
130 μl 50 mM deoxycholic acid solution pH 8.5 (final 0.5 mM)
6.5 μl chloramphenicol solution (final 20 μg/ml)
100 μl L-arabinose dehydrogenase
(from Burkholderia vietnamiensis, recombinantly expressed in E. coli, 400 U/ml)
98 mg L-arabinose (final 50 mM)
2.0 ml E. coli lysate P450/PtR/Ptx (see example 4)
9.6 ml dH₂O

The reactions were set up in 50 ml unbaffled Erlenmeyer flasks and sealed with aluminium foil. The film was punctured in several places. It was gently shaken at 24° C. for 16 h. The substances present in the supernatant were extracted with ethyl acetate and evaporated. It was dissolved in a smaller volume of HPLC eluent (methanol/acetonitrile/H₂O+H₃βO₄ (pH=3.0); 40:30:33). Subsequently, the samples were analyzed using HPLC/RID (e.g., Agilent 1200 series;

column: Agilent ZORBAX Eclipse XDB-C18 4.6×150 mm, 5 μm; flow rate: 0.8 ml/min). One of the candidates examined (“P450_c866”) was able to hydroxylate deoxycholic acid to ursocholic acid. The deoxycholic acid used was almost completely converted in the process (>95%). The identity of the product ursocholic acid was verified by GC/MS analysis and by 2D NMR (data not shown).

In this example, a redox cofactor (NADH) is oxidized by the P450/Ptx/PtR reaction. The redox cofactor is reduced back to its original state by the cofactor regeneration (in this case, for example, using the sugar dehydrogenase arabinose dehydrogenase) (with arabinose being oxidized to arabinolactone/arabonic acid in this case). This enables the use of a substoichiometric amount of redox cofactor.

### Example 7: Examples: Conversion Dependent on Cofactor Concentration Conversion with Cofactor Recycling

Reaction mixture:
10 μl 10 mM NAD+
250 μl 100 mM TEA pH 8.2
25 μl glycerin (50%)
100 μl 50 mM deoxycholic acid solution pH 8.0 (final 10 mM)
6.75 mg cells (wW) as a 22.5% suspension in 100 mM TEA pH 8.0 and 25% glycerin
5 μl catalase (bovine, Sigma 4 mg/ml)
1.7 units xylitol/sorbitol dehydrogenase
25 μl 2M sorbitol (final 100 mM)
70.8 μl dH₂O

The reactions were set up in 1.5 ml screw-top bottles and sealed with lids with aluminium foil. The foil was punctured in several places. It was gently shaken at 24° C. for 18 h.

The recovery of NADH was effected by sorbitol or, respectively, xylitol dehydrogenase in the presence of sorbitol and NAD⁺.

200 μl of the reaction batch was diluted with 600 μl acetonitrile/5 μl H₃βO₄ (50%) and incubated at 55° C. for 15 minutes. Subsequently, the samples were centrifuged at 20817 rcf for 5 minutes and analyzed using HPLC/DAD (e.g., Agilent 1200 series; column: Merck Purospher STAR RP-18e 125×4 mm or Agilent Zorbax XDB-C₈ mm 150×4.6 mm, 3.5 μm, 5 μm, flow rate: 1.5 ml/min, gradient H₂O+H₃βO₄ (pH=2.6)/acetonitrile).

The deoxycholic acid used was converted quantitatively (100% conversion) to ursocholic acid under the above-mentioned conditions. The identity of the product ursocholic acid was verified by GC/MS analysis and by 2D NMR.

In this example, a redox cofactor (NADH) obtained by the cofactor recycling system sorbitol/xylitol dehydrogenase/sorbitol/NAD⁺ is used for the hydroxylation reaction. However, other systems for cofactor recycling can also be used (see the following table).

| Regeneration | | | | | | DOCA |
|---|---|---|---|---|---|---|
| enzyme/ | μl lysate | redox | concentration | substrate | concentration | conversion |
| oxidoreductase | oxidoreductase | cofactor | [mM] | oxidoreductase | [mM] | [%] |
| xylitol/sorbitol | 1.7 | NAD+ | 0.2 | sorbitol | 100 | 100% |
| dehydrogenase | | | | | | |
| arabinose | 3 | NADH | 0.5 | arabinose | 50 | 95% |
| dehydrogenase | | | | | | |
| 12α-hydroxy- | 4 | NAD+ | 0.77 | cholic acid^{[1]} | 49 | 28.5 |
| steroid | | | | | | |
| dehydrogenase | | | | | | |
| 7α-hydroxy- | 4 | NAD+ | 0.77 | cholic acid^{[1]} | 49 | 17.2% |
| steroid | | | | | | |
| dehydrogenase | | | | | | |
| 12α-hydroxy- | 5/2.3 | NAD+ | 0.28 | cholic acid^{[2]} | 51.5 | 91% |
| steroid | | | | | | |
| dehydrogenase/ | | | | | | |
| 7α-hydroxy- | | | | | | |
| steroid | | | | | | |
| dehydrogenase | | | | | | |
| 12α-hydroxy- | 5/2.3/1.3 | NAD+ | 0.28 | cholic acid^{[2]} | 51.5 | 100% |
| steroid | | | | | | |
| dehydrogenase/ | | | | | | |
| 7α-hydroxy- | | | | | | |
| steroid | | | | | | |
| dehydrogenase/ | | | | | | |
| oxidase | | | | | | |
| NADH dependent | 6 | NAD+ | 0.19 | 2.3-butanediol | 427 | 98% |
| alcohol | | | | | | |
| dehydrogenase/ | | | | | | |
| 6.75 mg | | | | | | |
| hydroxylase | | | | | | |
| NADPH dependent | 2 | NADP+ | 0.19 | 2-propanol | 909 | 100% |
| alcohol | | | | | | |
| dehydrogenase/ | | | | | | |
| 6.75 mg | | | | | | |
| hydroxylase | | | | | | |
| formate | 4 | NAD+ | 0.82 | Na formate | 204 | 21.5% |
| dehydrogenase | | | | | | |

^{[1]}Used as a 20% cholic acid solution
^{[2]}Used as a bile acid solution (237 mM cholic acid; 45 mM deoxycholic acid)

### Example 8: Quantitative LCA (Lithocholic Acid) Conversion

Reaction mixture:
10 μl 10 mM NAD+
250 μl 100 mM TEA pH 8.2
10 mM (final) lithocholic acid
9.2 mg cells (wW) as a 22.5% suspension in 100 mM TEA pH 8.0 and 25% glycerin
5 μl catalase (bovine, Sigma 4 mg/ml)
1.7 units xylitol/sorbitol dehydrogenase
25 μl 2M sorbitol (final 100 mM)
176 μl dH₂O

The reactions were set up in 1.5 ml screw-top bottles and sealed with lids with aluminium foil. The foil was punctured in several places. It was gently shaken at 24° C. for 18 h.

The recovery of NADH was effected by sorbitol or, respectively, xylitol dehydrogenase in the presence of sorbitol and NAD+.

200 μl of the reaction batch was diluted with 600 μl acetonitrile/5 μl H₃βO₄ (50%) and incubated at 55° C. for 15 minutes. Subsequently, the samples were centrifuged at 20817 rcf for 5 minutes and analyzed using HPLC/DAD (e.g., Agilent 1200 series; column: Merck Purospher STAR RP-18e 125×4 mm or Agilent Zorbax XDB-C₈ mm 150×4.6 mm, 3.5 μm, 5 μm; flow rate: 1.5 ml/min, gradient H₂O+H₃βO₄ (pH=2.6)/acetonitrile).

Under the above-mentioned conditions, only ursodeoxycholic acid was detected after the conversion.

### Example 9: Conversion of LCA (Lithocholic Acid) to Ursodeoxycholic Acid in the Presence of Organic Solvents

At first, the solubility limits of LCA and UDCA in various organic solvents were determined. For this purpose, 10 mg or, respectively, 100 mg of LCA or UDCA was placed in a 15 mL flask. The organic solvent was added in increments of 100 μL, and the mixture was treated by shaking in a vortex shaker and optionally in an ultrasonic bath. It was visually assessed as to whether a clear solution was present.

The following table summarizes the solubility limits determined for the analyzed solvents:

| Solvent | UDCA [mg/ml] | LCA [mg/ml] |
|---|---|---|
| methanol | >100.0 | 28.6 |
| ethanol | 54.5 | 32.3 |
| isopropanol (IPA) | 47.5 | 33.7 |
| ethylene glycol | 16.6 | <1.0 |
| ethyl acetate (EtOAc) | 2.9 | 2.9 |
| methyl isobutyl ketone (MIBK) | 4.4 | 4.3 |
| methyl isobutyl alcohol (MIBA) | 35.7 | 33.7 |
| DMSO | >500.0 | 103.8 |
| DMF | >500.0 | 248.0 |
| DMA | >500.0 | >500.0 |
| hexane/EtOAc + 0.01% trifluoroacetic | <0.8 | 1.4 |
| acid (TFA) | | |
| hexane/IPA 8:2 + 0.01% TFA | 4.0 | 6.7 |
| hexane/IPA 7:3 + 0.02% TFA | 12.0 | 10.6 |
| cyclohexane | <0.7 | <0.6 |
| toluene | <1.0 | <1.0 |
| THF | 259.0 | 128.5 |
| Me—THF | 63.4 | 64.8 |
| dioxane | 92.6 | 51.4 |
| Et₂O | 2.0 | 3.4 |
| i Pr₂O | <0.5 | 0.9 |
| 2-butanol | 53.0 | 34.3 |
| 2-butanone | 10.0 | 10.0 |
| acetone | 8.8 | 4.9 |
| CHCl₃ | 2.9 | 4.9 |
| CHCl₃/phenol | 21.3 | 18.2 |
| CH₂Cl₂ | 1.1 | 1.4 |

The conversion of LCA to UDCA in the presence of the solvents was determined as indicated in the following tables:

| | conc. | conc. | | |
|---|---|---|---|---|
| | LCA | LCA | | |
| Solvent | [mg/ml] | [%] | conversion* | |
| EtOH | 4.3 | 0.4 | 4% | protic |
| MIBA | 4.3 | 0.4 | 3% | |
| DMSO | 4.4 | 0.4 | 31% | aprotic |
| | 4.8 | 0.5 | 32% | |
| | 5.6 | 0.6 | 41% | |
| | 7.4 | 0.7 | 48% | |
| | 11.0 | 1.1 | 52% | |
| | 14.8 | 1.5 | 45% | |
| | 17.4 | 1.7 | 9% | |
| | 21.8 | 2.2 | 7% | |
| DMF | 4.8 | 0.5 | 30% | |
| | 5.6 | 0.6 | 63% | |
| | 7.9 | 0.8 | 43% | |
| | 10.0 | 1.0 | 70% | |
| | 11.3 | 1.1 | 48% | |
| | 16.9 | 1.7 | 42% | |
| | 22.5 | 2.3 | 35% | |
| | 28.2 | 2.8 | 28% | |
| DMA | 3.7 | 0.4 | 22% | |
| | 5.9 | 0.6 | 41% | |
| | 7.9 | 0.8 | 44% | |
| | 11.9 | 1.2 | 51% | |
| | 13.6 | 1.4 | 53% | |
| | 16.5 | 1.7 | 50% | |
| | 22.4 | 2.2 | 6% | |
| | 30.0 | 3.0 | 4% | |
| | 37.4 | 3.7 | 3% | |
| Me—THF | 4.8 | 0.5 | 8% | |
| THF | 4.8 | 0.5 | 7% | |
| | 9.9 | 1.0 | 12% | |
| | 13.2 | 1.3 | 14% | |
| | 19.7 | 2.0 | 17% | |

### Example 10: Conversion of Lithocholic Acid to Ursodeoxycholic Acid with Increased Substrate Concentration in the Presence of an Aprotic Solvent

Reaction mixture:
10 μl 10 mM NAD+
250 μl 200 mM TEA pH 8.4 with 10.8% glycerin
25 μl 500 mM lithocholic acid in DMF
30 mg cells (wW) as a 30% suspension in 100 mM TEA pH 9.0
5 μl catalase (bovine, Sigma 4 mg/ml)
1.7 units xylitol/sorbitol dehydrogenase
25 μl 2M sorbitol (final 100 mM)
73 μl dH₂O

The reactions were set up in 1.5 ml screw-top bottles and sealed with lids with aluminium foil. The foil was punctured in several places. It was gently shaken at 24° C. for 18 h.

The recovery of NADH was effected by sorbitol or, respectively, xylitol dehydrogenase in the presence of sorbitol and NA⁺.

The reaction batch was completely evaporated in a stream of air and redissolved with 1.1 ml IPA+0.5% TFA. Subsequently, the samples were centrifuged at 20817 rcf for 5 minutes, and the supernatant was analyzed by HPLC/RID (e.g., Agilent 1200 series; column: Phenomenex Luna® Silica 100 Å, 250×4.6 mm, 5 μm; flow rate: 1.0 ml/min, n-hexane/IPA 4:1+0.05% TFA isocratic).

Under the above-mentioned conditions, 70% ursodeoxycholic acid was detected after the conversion.

1. A method of preparing a steroid having the general formula (I): 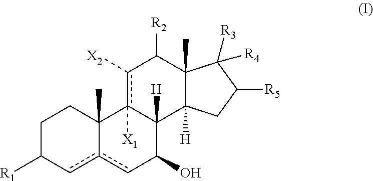
wherein
X₁ and X₂ are independently H, Cl, F, Br, I, CF₃, a C₁ to C₆ alkyl radical, OH, a C₁ to C₆ alkoxy radical, CN, NO₂, N(R₆)₂, an epoxy group, CHO, or a CO₂R₆ radical, wherein
R₆ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH(CH₃)₂, —C(O)C(CH₃)₃, —C(O)Ph, or —C(O)CH₂Ph,
R₁ and R₂ are independently H, OH, OR₇ or O, wherein
R₇ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH(CH₃)₂, —C(O)C(CH₃)₃, —C(O)Ph, or —C(O)CH₂Ph,
R₃ is H, OH, OR₈, a C₁ to C₁₀ alkyl radical, a C₁ to C₁₀ alkenyl radical, —CHO, —C(O)(CH₃), —C(O)(CH₂OH—), —CH(CH₃)C(O)CH₃, —C(CH₃)((CH₂)₂CO₂R₉), or —CH(CH₃)((CH₂)₂CONHR₉), wherein
R₈ is —C(O)H, —C(O)(CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, C(O)CH₂CH(CH₃)₂, —C(O)C(CH₃)₃, —C(O)Ph, or —C(O)CH₂Ph, and
R₉ is —CH₃, —CH₂COOH, —CH₂CH₃, —CH(CH₃)₂, —(CH₂)₂CH₃, —(CH₂)₂SO₃H, C(CH₃)₃, —(CH₂)₃CH₃, —CH(CH₃)C₂CH₃, —CH₂CH₂(CH₃)₂, an aryl group, or an alkylaryl group,
R₄ is H, OH, or —OR₁₀, wherein
R₁₀ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH(CH₃)₂, —C(O)C(CH₃)₃, —C(O)Ph, or —C(O)CH₂Ph, and
R₅ is H, CF₃, a C₁ to C₆ alkyl radical, a C₁ to C₆ alkenyl radical, OH, O, or a C₁ to C₆ alkoxy radical, wherein the dashed line denotes an optional double bond, with the proviso that the B ring has no double bond if the A ring has a C4-C5 double bond, and the C ring has no double bond if X₁ and X₂ form an epoxy group, or
wherein the steroid having general formula (I) is selected from the group consisting of 3α,7β,12α-trihydroxy-5β-cholane-24-acid, 3α,7β,12β-trihydroxy-5β-cholane-24-acid, 3β,7β,12α-trihydroxy-5β-cholane-24-acid, 3β,7β,12β-trihydroxy-5β-cholane-24-acid, 7β,12α-dihydroxy-3-keto-5β-cholane-24-acid, 7β,12β-dihydroxy-3-keto-5β-cholane-24-acid, 3α,7β-dihydroxy-12-keto-5β-cholane-24-acid, 3β,7β-dihydroxy-12-keto-5β-cholane-24-acid, 7β-hydroxy-3,12-diketo-5β-cholane-24-acid, 3α,7β-dihydroxy-5β-cholane-24-acid, 7β-hydroxy-3-keto-5β-cholane-24-acid and 3β,7β-dihydroxy-5β-cholane-24-acid,
the method comprising the step of converting a 7-deoxysteroid selected from the group consisting of 3α,12α-dihydroxy-5β-cholane-24-acid, 3α,12β-dihydroxy-5β-cholane-24-acid, 3β,12α-dihydroxy-5β-cholane-24-acid, 3β,12β-dihydroxy-5β-cholane-24-acid, 3β-hydroxy-12-keto-5β-cholane-24-acid, 3-keto, 12β-hydroxy-5β-cholane-24-acid, 3α-hydroxy-12-keto-5β-cholane-24-acid, 3-keto, 12α-hydroxy-5β-cholane-24-acid, 3,12-diketo-5β-cholane-24-acid, 3α-hydroxy-5β-cholane-24-acid, 3-keto-5β-cholane-24-acid and 3β-hydroxy-5β-cholane-24-acid or having the general formula (II) with a cytochrome P450 hydroxylase or a functional variant thereof in the presence of at least one redox partner system and a system for regenerating the redox partner system: 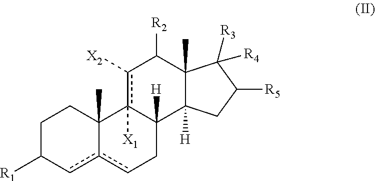
wherein the cytochrome P450 hydroxylase comprises an amino acid sequence which is at least 90% identical to the amino acid sequence of SEQ ID No. 1 or SEQ ID No. 2.

2. A method according to claim 1, wherein
X₁, X₂, R₄ and R₅ are H, and
R₁ und R₂ are independently H, OH, OR₇ or O, wherein
R₇ is —C(O)H, —C(O)CH₃, —C(O)CH₂CH₃, —C(O)(CH₂)₂CH₃, —C(O)CH(CH₃)₂, —C(O)(CH₂)₃CH₃, —C(O)CH(CH₃)CH₂CH₃, —C(O)CH₂CH(CH₃)₂, —C(O)C(CH₃)₃, —C(O)Ph, or —C(O)CH₂Ph,
R₃ is a C₁ to C₁₀ alkenyl radical, —CH(CH₃)((CH₂)₂CO₂R₉), or —CH(CH₃)((CH₂)₂CONHR₉), wherein
R₉ is —CH₃, —CH₂COOH, —CH₂CH₃, —CH(CH₃)₂, —(CH₂)₂CH₃, —(CH₂)₂SO₃H, C(CH₃)₃, —(CH₂)₃CH₃, —CH(CH₃)CH₂CH₃, —CH₂CH(CH₃)₂, an aryl group, or an alkylaryl group.

3. A method according to claim 1, wherein the aryl group is selected from the group consisting of a phenyl radical, a phenyl radical substituted with F, Cl, Br, NO₂ or CH₃, and a heteroaryl.

4. A method according to claim 1, wherein the alkylaryl group is selected from the group consisting of a benzyl group, a halogenated benzyl group, wherein the halogen is F, C₁ or Br, and a benzyl group substituted with NO₂.

5. A method according to claim 1, wherein R₁ is OH, R₂ is O, or OH, R₃ is —CH(CH₃)((CH₂)₂CO₂R₉), R₄ is H, and R₅ is H.

6. (canceled)

7. (canceled)

8. A method according to claim 1, wherein the cytochrome P450 hydroxylase is encoded by a nucleic acid which is at least 90% identical to the nucleic acid sequence SEQ ID No. 3, SEQ ID No. 4, SEQ ID No. 5, or SEQ ID No. 6.

9. A method according to claim 1, wherein the at least one redox partner system comprises:
(i) ferredoxin, ferredoxin reductase, and NAD(P)H;
(ii) cytochrome P450 reductase and NAD(P)H; or
(iii) NAD(P)H.

10. A method according to claim 9, wherein the ferredoxin is selected from the group consisting of adrenodoxins, putidaredoxins, and flavodoxins.

11. A method according to claim 9, wherein the at least one ferredoxin reductase is selected from the group of flavodoxin reductases and putidaredoxin reductase.

12. A method according to claim 1, wherein the system for the regeneration of the redox partner system comprises at least one oxidoreductase and at least one substrate of the at least one oxidoreductase.

13. A method according to claim 12, wherein the at least one oxidoreductase is selected from the group consisting of oxidoreductase (EC: 1.1.1), aldehyde dehydrogenase (EC: 1.2.1), amino acid dehydrogenase (EC: 1.4.1), flavin reductase (EC: 1.5.1), transhydrogenase (EC: 1.6.1), nitrite reductase (EC: 1.7.1) and phosphonate dehydrogenase (EC: 1.20.1), preferably selected from the group consisting of alcohol dehydrogenase, hydroxysteroid dehydrogenase, phosphite dehydrogenase, and sugar dehydrogenase.

14. A method according to claim 12, wherein the at least one oxidoreductase is selected from the group consisting of glucose dehydrogenase, glucose-6-phosphate dehydrogenase, arabinose dehydrogenase, xylose dehydrogenase, sorbitol dehydrogenase, xylitol dehydrogenase, 12α-hydroxysteroid dehydrogenase, 7α-hydroxysteroid dehydrogenase, 20α-hydroxysteroid dehydrogenase, 17β-hydroxysteroid dehydrogenase, 17α-hydroxysteroid dehydrogenase, 3α-hydroxysteroid dehydrogenase, 3β-hydroxy-delta5 dehydrogenase, 11β-hydroxysteroid dehydrogenase, and formate dehydrogenase.

15. A method according to claim 12, wherein the at least one substrate of the at least one oxidoreductase is selected from the group consisting of arabinose, xylose, glucose, sorbitol, xylitol, cholane-24-acid, 3α,12α-dihydroxycholane-24-acid-2,3-butanediol, acetoin, 2-propanol, glutamates, ethanol, phosphonates, phosphites, nitrites, 4-methyl-2-pentanol, 2-butanol, 2-octanol, cyclohexanol, ethanediol, 1,2-propanediol, 1-propanol, 1-butanol, and formate, 3-hydroxybutanoate.

16. A method according claim 1, wherein the method is performed in the presence of at least one organic solvent.

17. A method according to claim 16, wherein the at least one organic solvent is a protic or aprotic solvent.

18. A method according to claim 16, wherein the at least one organic solvent is selected from the group consisting of dimethylformamide (DMF), dimethyl sulfoxide (DMSO), and dimethylacetamide (DMA).

19. The method according to claim 1, wherein the cytochrome P450 hydroxylase comprises an amino acid sequence which is 100% identical to the amino acid sequence of SEQ ID No. 1 or SEQ ID No. 2

20. A method according to claim 17, wherein the at least one organic solvent is an aprotic solvent.
